(19) United States
(12) Patent Application Publication
(10) Pub. No.: US 2009/0149494 A1
(43) Pub. Date: Jun. 11, 2009

(54) THERAPEUTIC USE OF IMIDAZO[1,2-a]PYRIDINE-2-CARBOXAMIDE DERIVATIVES

(75) Inventor: Jean-Francois PEYRONEL, Antony (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/337,018

(22) Filed: Dec. 17, 2008

Related U.S. Application Data
(63) Continuation of application No. PCT/FR2007/001127, filed on Jul. 3, 2007.

(30) Foreign Application Priority Data
Jul. 3, 2006 (FR) 0606013

(51) Int. Cl. A61K 31/437; A61P 25/00; A61P 29/00; A61P 35/00

(52) U.S. Cl. 514/300

(57) ABSTRACT

The present invention is related to the use of a compound of formula (I):

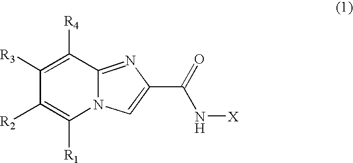

Wherein R₁, R₂, R₃, R₄ and X are as defined herein, or an addition salt with an acid, for the preparation of a medicament for treating or preventing diseases in which the NOT receptor is involved.

This application is a Continuation of International Application No. PCT/FR2007/001127, filed Jul. 3, 2007, which is incorporated herein by reference in its entirety.

### FIELD OF THE INVENTION

The present invention relates to the therapeutic use of imidazo[1,2-α]pyridine-2-carboxamide derivatives in the treatment or prevention of diseases involving the Nurr-1 nuclear receptors, also known as NR4A2, NOT, TINUR, RNR-1 and HZF3.

### SUMMARY OF THE INVENTION

One subject of the present invention is the use of compounds corresponding to the formula (I):

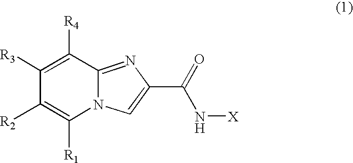

in which
R₁, R₂, R₃ and R₄ represent a hydrogen atom; and X represents a phenyl group optionally substituted with one or more groups chosen, independently of each other, from the following atoms or groups: halogen, (C₁-C₆)alkoxy, (C₁-C₆)alkyl, cyclo(C₁-C₆)alkyl(C₁-C₆)alkyl, cyclo(C₁-C₆)alkyl(C₁-C₆)alkoxy, NRaRb;
or R₂ is chlorine and X is a para-fluorophenyl;
or R₃ is a methyl and X is an unsubstituted phenyl group;
or R₁ is a methyl and X is an unsubstituted phenyl group;
R^{a} and R^{b} are, independently of each other, hydrogen or (C₁-C₆)alkyl, or form, with
the nitrogen atom, a 4- to 7-membered ring;
in the form of the base or of an acid-addition salt,
for the preparation of a medicament for treating or preventing diseases in which the NOT receptor is involved.

### DETAILED DESCRIPTION OF THE INVENTION

Among the compounds of formula (I) that are subjects of the invention, a first group of compounds is constituted by compounds for which:

R₃ is a methyl and X is an unsubstituted phenyl group;
or R₁ is a methyl and X is an unsubstituted phenyl group;
in the form of the base or of an acid-addition salt.

The compounds of formula (I) may exist in the form of bases or of acid-addition salts. Such addition salts form part of the invention.

These salts may be prepared with pharmaceutically acceptable acids, but the salts of other acids that are useful, for example, for purifying or isolating the compounds of formula (I) also form part of the invention.

The compounds of formula (I) may also exist in the form of hydrates or solvates, i.e. in the form of associations or combinations with one or more water molecules or with a solvent. Such hydrates and solvates also form part of the invention.

Among the compounds of formula (I) that are subjects of the invention, mention may be made especially of the following compounds:
N-phenylimidazo[1,2-α]pyridine-2-carboxamide
6-chloro-N-(4-fluorophenyl)imidazo[1,2-α]pyridine-2-carboxamide
N-(2-bromo-4,6-difluorophenyl)-5-methylimidazo[1,2-α]pyridine-2-carboxamide
N-(4-azepan-1-ylphenyl)-5-methylimidazo[1,2-α]pyridine-2-carboxamide
N-(5-chloro-2,4-dimethoxyphenyl)-5-methylimidazo[1,2-α]pyridine-2-carboxamide
N-(2-methoxy-5-methylphenyl)-7-methylimidazo[1,2-α]pyridine-2-carboxamide
N-(3-fluorophenyl)-7-methylimidazo[1,2-α]pyridine-2-carboxamide
N-(2-fluorophenyl)-7-methylimidazo[1,2-α]pyridine-2-carboxamide
N-(4-fluorophenyl)-7-methylimidazo[1,2-α]pyridine-2-carboxamide
N-(2,5-diethoxyphenyl)-7-methylimidazo[1,2-α]pyridine-2-carboxamide
N-(2,4-dimethoxyphenyl)-7-methylimidazo[1,2-α]pyridine-2-carboxamide
N-(3,5-dimethoxyphenyl)-7-methylimidazo[1,2-α]pyridine-2-carboxamide
N-(3-methoxyphenyl)-7-methylimidazo[1,2-α]pyridine-2-carboxamide
N-(2-ethoxyphenyl)-7-methylimidazo[1,2-α]pyridine-2-carboxamide
7-methyl-N-(2-methylphenyl)imidazo[1,2-α]pyridine-2-carboxamide
N-(4-methoxyphenyl)-7-methylimidazo[1,2-α]pyridine-2-carboxamide
7-methyl-N-(2-piperidin-1-ylphenyl)imidazo[1,2-α]pyridine-2-carboxamide
N-(2,5-dimethoxyphenyl)-7-methylimidazo[1,2-α]pyridine-2-carboxamide
N-(2-methoxyphenyl)-7-methylimidazo[1,2-α]pyridine-2-carboxamide
N-(4-aminophenyl)-7-methylimidazo[1,2-α]pyridine-2-carboxamide
N-(2-fluorophenyl)imidazo[1,2-α]pyridine-2-carboxamide
N-(4-piperidin-1-ylphenyl)imidazo[1,2-α]pyridine-2-carboxamide
N-(3-chloro-4-fluorophenyl)imidazo[1,2-α]pyridine-2-carboxamide
N-(4-bromo-3-methylphenyl)imidazo[1,2-α]pyridine-2-carboxamide
N-(2-isopropyl-6-methylphenyl)imidazo[1,2-α]pyridine-2-carboxamide
N-(2-piperidin-1-ylphenyl)imidazo[1,2-α]pyridine-2-carboxamide
N-(3-ethylphenyl)imidazo[1,2-α]pyridine-2-carboxamide
N-(5-chloro-2-piperidin-1-ylphenyl)imidazo[1,2-α]pyridine-2-carboxamide
N-(2-chlorophenyl)imidazo[1,2-α]pyridine-2-carboxamide
6,8-dichloro-N-[4-(dimethylamino)phenyl]imidazo[1,2-α]pyridine-2-carboxamide.

In accordance with the invention, the compounds of general formula (I) may be prepared according to the process described in Scheme 1.

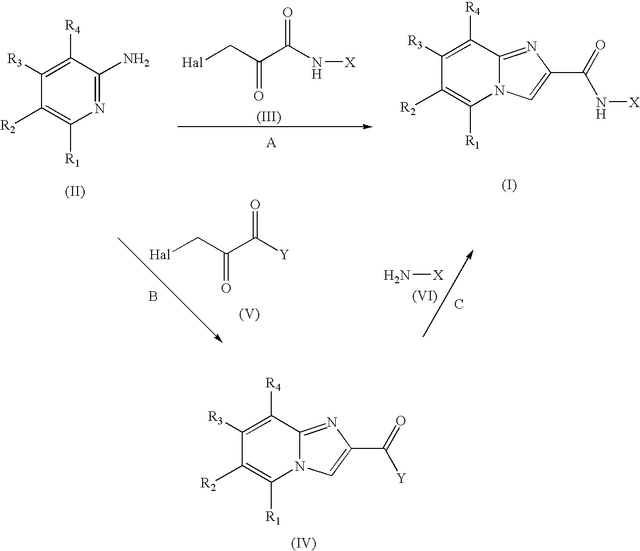

Route A consists in preparing the 2-aminopyridines of formula (TI) according to the methods known to those skilled in the art, and in forming the imidazo[1,2-α]pyridine ring by condensation with a 2-oxo-N-arylpropionamide derivative (III) in which Hal represents a chlorine, bromine or iodine atom and X is as defined previously, by analogy with the methods described by J-J. Bourguignon et al. in Aust. J. Chem. 1997, 50, 719-725 and by J. G. Lombardino, J. Org. Chem. (1965), 30(7), 2403 for example. The halo 2-oxo-N-arylpropionamide derivatives (III) may be obtained according to the method described by R. Kluger et al. in J. Am. Chem. Soc., (1984) 106(14), 4017.

The second synthetic route B-C consists in coupling an imidazopyridine-2-carboxylic acid or a derivative thereof, of formula (IV) in which Y is OH, halogen or (C₁-C₆)alkoxy, with an arylamine X—NH2 (VI) in which X is as defined previously, according to methods known to those skilled in the art. Thus, the acid may be converted beforehand into a reactive derivative thereof such as an acid halide, anhydride, mixed anhydride or activated ester, and then reacted with the amine (VI) in the presence of a base such as diisopropylethylamine, triethylamine or pyridine, in an inert solvent such as THF, DMF or dichloromethane. The coupling may also be performed in the presence of a coupling agent such as CDI, EDCI, HATU or HBTU under the same conditions, without isolating the reactive intermediate. Alternatively, the amine (VI) may be reacted with an ester of the acid of formula (IV) in the presence of a catalyst such as trimethylaluminum, according to the method of Weinreb, S. et al (Tet. Lett. (1977), 18, 4171) or zirconium tert-butoxide. The imidazopyridine-2-carboxylic acids and the derivatives thereof of formula (IV) may be obtained by condensing the appropriate 2-aminopyridines with a 3-halo-2-oxopropionic acid ester according to the method described by J. G. Lombardino in J. Org. Chem., 30(7), 2403 (1965), followed by deprotecting the ester to the acid and converting the acid, where appropriate, to a derivative thereof.

The products of formula (I) may be subjected, if desired and if necessary, in order to obtain products of formula (I) or to be transformed into other products of formula (I), to one or more of the following transformation reactions, in any order:
a) a reaction for the transformation of a hydroxyl function into an alkoxy function,
b) a reaction for the catalytic coupling of a halo derivative and of an organometallic derivative such as stannyl or boronyl, to introduce a methyl substituent,
c) a reaction for the protection of reactive functions,
d) a reaction for the removal of the protecting groups that may be borne by the protected reactive functions,
e) a salification reaction with a mineral or organic acid or with a base to obtain the corresponding salt,
f) a reaction for the resolution of racemic forms into enantiomers, said products of formula (I) thus obtained being, where appropriate, in any possible isomeric form: racemic mixtures, enantiomers and diastereoisomers.

In Scheme 1, the starting compounds and the reagents, when their mode of preparation is not described, are commercially available or are described in the literature, or else may be prepared according to methods that are described therein or that are known to those skilled in the art.

The compounds according to the invention underwent pharmacological tests to determine their modulatory effect on NOT.

### Evaluation of the In Vitro Activity on N2A Cells

The tests consisted in evaluating the in vitro activity of the compounds of the invention on a cell line (N2A) endogenously expressing the murine Nurr1 receptor and stably transfected with the NOT binding response element (NBRE) coupled to the luciferase reporter gene. The EC₅₀ values are between 0.01 and 1000 nM. The tests were performed according to the procedure described hereinbelow.

The cell line Neuro-2A is obtained from a standard commercial source (ATCC). The clone Neuro-2A was obtained from a spontaneous tumor originating from a strain of albino mice A by R. J Klebe et al. This line Neuro-2A is then stably transfected with 8NBRE-luciferase. The N2A-8NBRE cells are cultured to the point of confluence in 75 cm² culture flasks containing DMEM supplemented with 10% fetal calf serum, 4.5 g/L of glucose and 0.4 mg/ml of geneticin. After culturing for one week, the cells are recovered with 0.25% trypsin for 30 seconds and then resuspended in DMEM without phenol red, containing 4.5 g/L of glucose and 10% Hyclone defatted serum, and placed in white, transparent-based 96-well plates. The cells are deposited at a rate of 60 000 per well in 75 μL for 24 hours before adding the products. The products are applied in 25 μL and incubated for a further 24 hours. On the day of measurement, an equivalent volume (100 μL) of Steadylite is added to each well, and the wells are then left for 30 minutes to obtain complete lysis of the cells and maximum production of the signal. The plates are then measured in a microplate luminescence counter, after having been sealed with an adhesive film. The products are prepared in the form of a 10⁻² M stock solution, and then diluted in 100% of DMSO. Each concentration of product is prediluted in culture medium before incubation with the cells thus containing 0.625% final of DMSO.

For example, compounds 1 and 2 gave an EC₅₀ value of 5.5 nM and 17 nM, respectively.

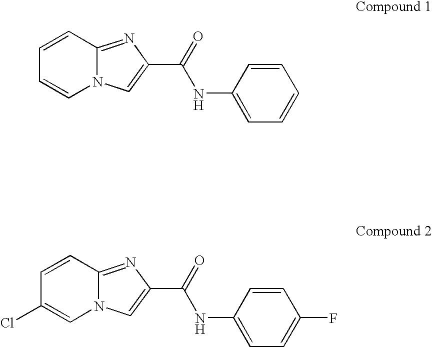

### Evaluation of the Binding to the Human NOT Receptor

The direct binding between compounds of the invention and the human NOT receptor was evaluated using the SPR (surface plasmon resonance) technique. In this test, the protein is immobilized covalently on the matrix and the test molecule is injected into the chamber containing the sensor chip. The signal is directly proportional to the amount of product bound to the protein. The binding tests were performed in a Biacore S51 machine (Biacore Inc., Piscataway N.J.). The entire GST-NOT protein (NOT-FL) was supplied by Invitrogen (PV3265). The domain for binding to the NOT ligand (His-Thr-NOT 329-598) was expressed and purified as described in Nature 423, 555-560. The two proteins, diluted to a concentration of 20 μg/ml in pH 5.0 acetate buffer containing 5 mM of DTT, were immobilized on a surface of carboxymethyl 5′ dextran (CM5 sensor chip, Biacore Inc.) via amine coupling according to the protocol recommended by Biacore, eluting with an HBS-N buffer (10 mM HEPES, 0.15 M NaCl, 3 mM EDTA, pH 7.4). Approximately 10 000-15 000 resonance units (R^{U}) of the proteins are captured on the surface of the sensor chip CM5. The stock solutions of the test compounds at 1.5 mM in DMSO are serially diluted in elution buffer (50 mM HEPES pH 8; 150 mM NaCl; 10 mM MgCl₂; 2% DMSO, 1 mM DTT) at concentrations ranging from 3.75 to 0.1 μM. Each concentration of product is injected at 4° C. for 1 minute at 30 μl/min. The dissociation was recorded for 5 minutes without any other surface regeneration procedure. The signals obtained are corrected by testing each concentration of product on a surface of unmodified dextran (blank). The signal due to the migration buffer product is subtracted from the total signal (“double referencing”), as is the effect of the DMSO. Analysis of the signals was performed using the Biacore S51 analysis software (version 1.2.1). The compounds are then classified as a function of their maximum binding level and of kinetic parameters of binding to the immobilized protein.

By way of example, compound 1 showed moderate affinity.

It is thus seen that the compounds according to the invention have a modulatory effect on NOT.

The compounds according to the invention may thus be used for the preparation of medicaments for their therapeutic application in the treatment or prevention of diseases involving the NOT receptors.

These medicaments find their therapeutic use especially in the treatment and prevention of neurodegenerative diseases, for instance Parkinson's disease, Alzheimer's disease, tauopathies (e.g. progressive supranuclear palsy, frontotemporal dementia, corticobasal degeneration, Pick's disease); multiple sclerosis; cerebral trauma, for instance ischemia and cranial trauma and epilepsy; psychiatric diseases, for instance schizophrenia, depression, substance dependency, and attention-deficit hyperactivity disorder; inflammatory diseases, for instance vascular pathologies, atherosclerosis, joint inflammations, arthrosis, rheumatoid arthritis, osteoarthritis, and allergic inflammatory diseases such as asthma, and finally for the treatment of osteoporosis and cancers.

These compounds may also be used as a treatment combined with grafts and/or transplantations of stem cells.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt of said compound, and also at least one pharmaceutically acceptable excipient.

Said excipients are chosen, according to the pharmaceutical form and the desired mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the salt thereof, may be administered in unit administration form, as a mixture with standard pharmaceutical excipients, to man and animals for the prophylaxis or treatment of the above complaints or diseases.

The appropriate unit forms of administration include oral forms such as tablets, soft or hard gel capsules, powders, granules and oral solutions or suspensions, sublingual, buccal, intratracheal, intraocular, intranasal or inhalation administration forms, topical, transdermal, subcutaneous, intramuscular or intravenous administration forms, rectal administration forms and implants. For topical application, the compounds according to the invention may be used in creams, gels, ointments or lotions.

By way of example, a unit administration form of a compound according to the invention in tablet form may comprise the following components:

| | | |
|---|---|---|
| Compound according to the invention | 50.0 | mg |
| Mannitol | 223.75 | mg |
| Croscarmellose sodium | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate; such dosages are not outside the context of the invention. According to the usual practice, the dosage that is appropriate for each patient is determined by the doctor according to the mode of administration and the weight and Response of said patient.

According to another of its aspects, the present invention also relates to a method for treating the pathologies indicated above, which comprises the administration, to a patient, of an effective dose of a compound according to the invention, or a pharmaceutically acceptable salt thereof.

We claim:

1. A method for treating or preventing a disease in which the NOT receptor is involved, in a patient in need thereof, comprising administering a pharmaceutically effective amount of a compound of formula (I): 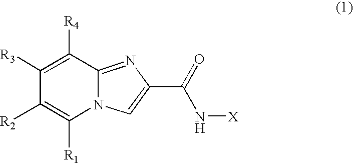
wherein:
R₁, R₂, R₃ and R₄ are hydrogen, and
X is phenyl optionally substituted with one or more groups chosen, independently of each other, from the group consisting of halogen, (C₁-C₆)alkoxy, (C₁-C₆)alkyl, cyclo(C₁-C₆)alkyl(C₁-C₆)alkyl, cyclo(C₁-C₆)alkyl(C₁-C₆)alkoxy, and NRaRb; or
R₁, R₃ and R₄ are hydrogen, R₂ is chlorine, and X is para-fluorophenyl; or
R₁, R₂ and R₄ are hydrogen, R₃ is methyl, and X is unsubstituted phenyl; or
R₂, R₃ and R₄ are hydrogen, R₁ is methyl, and X is unsubstituted phenyl; and
Ra and Rb are, independently, hydrogen or (C₁-C₆)alkyl, or
R^{a} and R^{b}, taken together with the nitrogen atom to which they are attached, form a 4- to 7-membered ring;

2. The method according to claim 1, wherein for the compound of formula (I),
R₁, R₂ and R₄ are hydrogen, R₃ is methyl, and X is unsubstituted phenyl; or
R₂, R₃ and R₄ are hydrogen, R₁ is methyl, and X is unsubstituted phenyl.

3. The method according to claim 1, wherein the disease in which the NOT receptor is involved is a neurodegenerative disease.

4. The method according to claim 1, wherein the disease in which the NOT receptor is involved is multiple sclerosis, cerebral trauma or epilepsy.

5. The method according to claim 1, wherein the disease in which the NOT receptor is involved is a psychiatric disease.

6. The method according to claim 1, wherein the disease in which the NOT receptor is involved is an inflammatory disease.

7. The method according to claim 1, wherein the disease in which the NOT receptor is involved is osteoporosis or cancer.

8. The method according to claim 1, wherein the disease in which the NOT receptor is involved is Parkinson's disease, Alzheimer's disease or tauopathy.

9. The method according to claim 1, wherein the disease in which the NOT receptor is involved is schizophrenia, depression, substance dependency or attention-deficit hyperactivity disorder.
